(19) United States
(12) Patent Application Publication
(10) Pub. No.: US 2015/0087546 A1
(43) Pub. Date: Mar. 26, 2015

(54) SEQUENCE, TECHNIQUE PLATFORM, AND METHOD FOR IN VITRO DETECTING CLOSTRIDIUM DIFFICILE RIBOTYPE 027

(71) Applicant: National Cheng Kung University, Tainan City (TW)

(72) Inventors: PEI-JANE TSAI, TAINAN CITY (TW); BO-YANG TSAI, TAINAN CITY (TW); YUAN-PIN HUNG, TAINAN CITY (TW); WEN-CHIEN KO, TAINAN CITY (TW)

(21) Appl. No.: 14/494,902

(22) Filed: Sep. 24, 2014

(30) Foreign Application Priority Data
Sep. 25, 2013 (TW) 102134634

(51) Int. Cl. C12Q 1/68

(52) U.S. Cl. CPC C12Q 1/689; C12Q 2600/16
USPC 506/9; 536/23.7; 506/16

(57) ABSTRACT

The invention relates to a sequence, a technique platform, and a method for in vitro detecting Clostridium difficile ribotype 027. The technology platform includes a pair of primers specific to C. difficile ribotype 027 and used for polymerase chain reaction (PCR), and primer sets as well as materials demanded for multiplex-PCR. The method comprises steps of obtaining a specimen DNA, in vitro amplifying the specimen DNA by the primer sets to detect whether the specimen is matched to characteristics of a target sequence of SEQ ID NO: 1. Specimen having a sequence matching to characteristics indicates that it comprises C. difficile ribotype 027. Accordingly, the present invention can achieve the aim at quickly confirming whether the specimen contains ribotype 027 strains.

### BACKGROUND OF THE INVENTION

1. Field of the Invention

The present invention relates to a sequence, a technique platform, and a method for in vitro detecting genotyping of epidemiological Clostridium difficile ribotype 027. Especially with a specific design of a pair of primers (#1 and #2) for a target sequence of SEQ ID NO:1 unique to C. difficile ribotype 027, the technique platform can rapidly and sensitively detect whether the specimen is matched to characteristics of a target sequence by polymerase chain reaction (PCR). Accordingly, the present invention can achieve the goal of quickly confirming whether the specimen contains ribotype 027 strains.

2. Description of Related Art

Clostridium difficile, initially published by Holdeman in 1977, is belonging to a family of Bacillaceae and a genus of clostridium; its genus name “clostridium” deriving from Greek refers to “spindle”, and the species name “difficile” deriving from Latin refers to difficulties in isolation and study (because it was resistant to early attempts at isolation and grew very slowly in culture). C. difficile is an anaerobic Gram-positive bacillus which is a zoonotic pathogen that may form spores outside the host body or in the environment. The spores resist to heating, drying and disinfectants. The spores often spread through acts of medical care or any live stocks. There are various strains of C. difficile with different toxicity existed in the world. Some of C. difficile strains do not cause disease by competitively inhibition of other gut flora. However, in the hospital, patients exposed to the usage of antibiotics may promote the opportunistic C. difficile infections by disruption the homeostasis of gut flora and lead C. difficile proliferation. The virulence of C. difficile primarily comes from its secretion of toxin A and toxin B, which can cause inflammation and damage to the intestinal mucosa. Accordingly, C. difficile may cause pseudomembranous colitis, severe diarrhea, sepsis, and even death to patients. Furthermore, mortality is substantially greater in patients with C. difficile infections involving strains producing higher toxin AIB and binary toxin, concerned the strains are PCR ribotype 027 with a deletion in the regulatory tcdC gene. Therefore, such genetic changes are presumably associated with an increased secretion of toxin A and toxin B.

Recently, an epidemiological change of C. difficile infection (CDI) has been associated with the appearance of a C. difficile ribotype 027 strain, which is known to have a high toxicity. Outbreaks caused by this ribotype 027 strain usually lead to high mortality and relapses rates. Therefore, specific identification of C. difficile will facilitate the understanding of the epidemiology of the infection. PCR ribotyping is the currently prevailing typing method. PCR ribotyping is performed by a PCR-based method to detect polymorphic sequences in the 16S-23S intergenic spacer region in C. difficile. The band-pattern result, fingerprinting, generated by this method is hard to compare between international laboratories. Till now, there is no diagnostic technique to directly determine whether it is C. difficile ribotype 027 or not. It must use the traditional PCR ribotyping method and then compare to the genetic fingerprinting of ribotype 027 reference strain to figure out whether is ribotype 027. Referring to FIG. 7, an electropherogram showed the result of detecting C. difficile by a traditional ribotyping method. There are disadvantages existed in use of traditional ribotyping method for differentiating what is the ribotype of each clinical C. difficile isolates, i.e. necessity for preparing many known ribotyping strains or standard strains as a typing standard basis in advance, requirement of a powerful software and computer for fingerprinting comparison, low reproducibility in the laboratory and complicated procedures.

### SUMMARY OF THE INVENTION

In view of the above-mentioned problems, the object of the present invention is to provide a sequence, a technique platform, and a method for in vitro detecting genotyping of epidemiological Clostridium difficile ribotype 027. The technique platform detects whether a specimen is matched to characteristics of a target sequence by using a #1 primer and a #2 primer for polymerase chain reaction (PCR). Therefore, the present invention can achieve the goal of quickly confirming whether the specimen contains ribotype 027 strain.

Disclosed herein are a sequence, a technique platform, and a method for detecting C. difficile ribotype 027. The technique platform and the method are used for purpose of detecting an in vitro purified and isolated target sequence; wherein the target sequence can be a DNA sequence comprising at least 85% sequence homology with SEQ ID NO:1, preferably at least 90% homology, more preferably at least 95% homology or 100% homology with SEQ ID NO:1.

A technique platform for in vitro detecting C. difficile ribotype 027 comprises: a pair of primers including a #1 primer and a #2 primer specific to C. difficile ribotype 027 for conducting polymerase chain reaction (PCR); and primer sets and materials demanded for multiplex-PCR. In the foregoing description, the #1 primer includes a sequence of SEQ ID NO:2 and the #2 primer includes a sequence of SEQ ID NO:3, and both primers are designed for the sequence of SEQ ID NO:1 of C. difficile ribotype 027.

A method for in vitro detecting C. difficile ribotype 027 is also revealed, comprising the steps of obtaining a specimen DNA; and then in vitro amplifying the specimen DNA by primer sets to detect whether the specimen is matched to characteristics of a target sequence of SEQ ID NO: 1. Specimen having a sequence matching to characteristics means that it comprises C. difficile ribotype 027. In the foregoing description, the characteristic of the target sequence of SEQ ID NO: 1 comprises a sequence or a length of the sequence.

According to an embodiment of the present invention, the specimen is selected from infected stools or C. difficile isolates.

### BRIEF DESCRIPTION OF THE DRAWINGS

FIG. 1 is a flowchart of a method according to the present invention.

FIG. 2-A is an electropherogram showing the ribotyping results of thirty-one standard strains. Only four of ribotype 027 strains (R20291, BAA 1805, BI-1, and BI-7) have a DNA product consistent with the fragment size of the target sequence.

FIG. 2-B is an electropherogram showing the ribotyping results of twenty standard strains. Non-ribotype 027 strain doesn't have a DNA product consistent with the fragment size of the target sequence.

FIG. 3-A is an electropherogram showing the ribotyping results of No. 1-41 clinical strains. Only ribotype 027 strains (BAA 1805 and R20291) have a DNA product consistent with the fragment size of the target sequence.

FIG. 3-B is an electropherogram showing the ribotyping results of No. 42-73 clinical strains. Only ribotype 027 strains (R20291) have a DNA product consistent with the fragment size of the target sequence.

FIG. 3-C is an electropherogram showing the ribotyping results of No. 74-119 clinical strains. Only ribotype 027 strains (BAA 1805 and R20291) have a DNA product consistent with the fragment size of the target sequence.

FIG. 3-D is an electropherogram showing the ribotyping results of No. 120-149 clinical strains. Only ribotype 027 strains (BAA 1805 and R20291) have a DNA product consistent with the fragment size of the target sequence. Only ribotype 027 strains (BAA 1805 and R20291) have a DNA product consistent with the fragment size of the target sequence.

FIG. 3-E is an electropherogram showing the ribotyping results of No. 150-210 clinical strains. Only ribotype 027 strains (BAA 1805 and R20291) have a DNA product consistent with the fragment size of the target sequence.

FIG. 3-F is an electropherogram showing the ribotyping results of No. 211-273 clinical strains. Only ribotype 027 strains (BAA 1805 and R20291) have a DNA product consistent with the fragment size of the target sequence.

FIG. 3-G is an electropherogram showing the ribotyping results of No. 274-334 clinical strains. Only ribotype 027 strains (BAA 1805 and R20291) have a DNA product consistent with the fragment size of the target sequence.

FIG. 3-H is an electropherogram showing the ribotyping results of No. 335-380 clinical strains. Only ribotype 027 strains (BAA 1805 and R20291) have a DNA product consistent with the fragment size of the target sequence.

FIG. 3-I is an electropherogram showing the ribotyping results of No. 381-414 clinical strains. These known clinical strains don't have a DNA product consistent with the fragment size of the target sequence.

FIG. 4 is an electropherogram showing the ribotyping results of different C. difficile strains. Only ribotype 027 strains (R20291, BAA 1805, BI-1, and BI-7) have a DNA product consistent with the fragment size of the target sequence.

FIG. 5 is an embodiment of a toxin genotyping result of C. difficile. The result can be used for a basis to rapidly detect toxigenic C. difficile.

FIG. 6 is an electropherogram showing the results of toxin genotyping and ribotyping of different C. difficile strains.

FIG. 7 is an electropherogram showing the result of detecting C. difficile by a traditional ribotyping method.

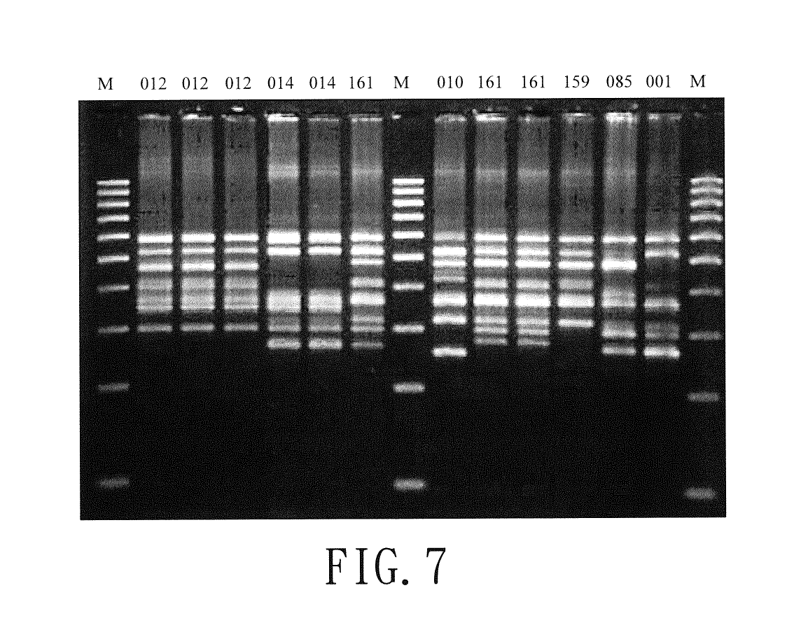

### DETAILED DESCRIPTION OF THE PREFERRED EMBODIMENT

A purified and isolated target sequence for in vitro detecting Clostridium difficile ribotype 027 is revealed. For instance, it can be a DNA sequence comprising at least 85% sequence homology with SEQ ID NO:1 (as shown on the sequence listing), preferably at least 90% homology, more preferably at least 95% homology or 100% homology with SEQ ID NO:1.

First, a technique platform for in vitro detecting C. difficile ribotype 027 is revealed, comprising:

a pair of primers including a #1 primer and a #2 primer specific to C. difficile ribotype 027 for conducting polymerase chain reaction (PCR), wherein the #1 primer includes a sequence of SEQ ID NO:2 and the #2 primer includes a sequence of SEQ ID NO:3, and both primers are designed for the sequence of SEQ ID NO:1 of C. difficile ribotype 027; and

primer sets and materials demanded for multiplex-PCR.

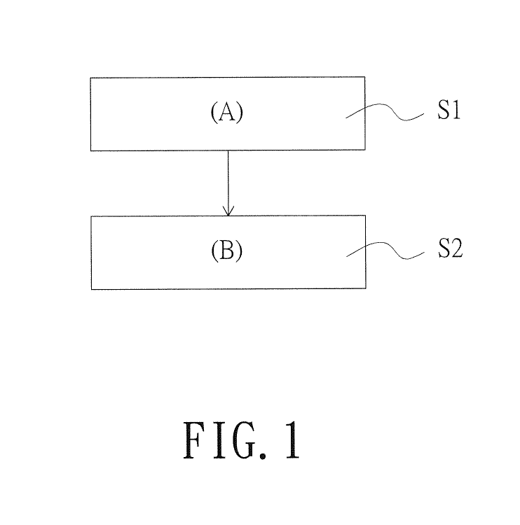

Referring to FIG. 1, a flowchart of a method according to the present invention is disclosed. A method for in vitro detecting C. difficile ribotype 027 comprises the steps of:

(A) (S1) obtaining a specimen DNA, wherein the specimen is the one which has been identified as C. difficile, with C. difficile toxins or a combination thereof; and

(B) (S2) in vitro amplifying the specimen DNA by primer sets according to the abovementioned technique platform (can be, but not limit to a kit) to detect whether the specimen is matched to characteristics of a target sequence of SEQ ID NO: 1, e.g. a sequence or a length of the sequence (2,981 bp). Specimen having a sequence matching to characteristics indicates that it comprises C. difficile ribotype 027.

Hereinafter, an exemplary embodiment of the present invention will be described in detail with reference to the accompanying drawings.

Briefly, the present invention relates to a technique platform for detecting C. difficile ribotyp 027 based on the specific sequences. This technology platform includes: the primer set which can be used by polymerase chain reaction (PCR) specifically to detect C. difficile ribotype 027 and the algorithmic procedure. Here, a multiplex PCR assay is developed for C. difficile detection and epidemiological typing for the ribotype 027. Mutiplex PCR were performed after obtain DNA from clinical stool specimen. Three sets of primers were designed to detect toxigenic C. difficile and C. difficile ribotype 027 will be identified by amplification with the primers which specifically matched to the target sequence SEQ ID NO: 1. The present inventors identified a specific gene loci and developed a one-step, rapid and sensitive diagnostic platform for the detection of C. difficile ribotype 027.

### Example 1

### Obtaining a Specimen DNA and Confirming the Toxigenic C. difficile Isolates

Forty standard strains with known ribotype were obtained from the Prof. Goldstein's lab of University of California in Los Angeles (UCLA), and eleven standard strains were purchased from the CCUG (Culture Collection, University of Goteborg, Sweden). Furthermore, 404 clinical C. difficile strains were isolated and obtained from patients in the Tainan Hospital. All abovementioned bacteria strains were stored at −80° C.

Anaerobic culture was performed by plating each bacteria strain onto a CDC anaerobe 5% sheep blood agar and incubated in an anaerobic tank at 37° C. for 48 hours. Then a colony of each strain was selected and inoculated in a sealed centrifuge tube including 50 ml culture medium therein (brain heart infusion (BHI) broth) for anaerobic incubation at 37° C. for 48 hours. The culture medium comprises 37 g/L brain heart infusion, 5 g/L yeast extraction, and 1 g/L L-cysteine.

After centrifugation of each centrifuge tube containing various strains at 2,500 g for 10 minutes, the supernatant was removed and the pellet was suspended in 1 mL PBS buffer. Then 200 μL buffer containing each strain therein was used in DNA extraction according to manufacturer's instruction of Genomic DNA Mini Kit (Geneaid).

Furthermore, toxin genotyping was conducted by means of multiplex-PCR to detect 16s rDNA, tcdA, tcdB, cdtA, cdtB, and tcdC genes of C. difficile. Sequences of the primers specific to six genes were listed in Table 1 and the parameters for the amplification were listed in the followings. The parameters were: (1) denature template at 94° C. for 5 min; (2) amplify template for 35 cycles of 1 min at 94° C., 1 min at 55° C., and 1 min at 72° C.; and (3) extension (or elongation) at 72° C. for 5 min; the final PCR products were stored at 4° C. A 2% agarose gel was used for DNA electrophoresis (100 volts for 40 min), and then the gel was stained with ethidium bromid (EtBr) for analysis.

Table 1

| Gene | | |
|---|---|---|
| name | Primer | Sequence (5'→3') |
| 16S | PS13 | GGAGGCAGCAGTGGGGAATA |
| rDNA | PS14 | TGACGGGCGGTGTGTACAAG |
| tcdA | f3345 | GCATGATAAGGCAACTTCAGTGGTA |
| | R3969 | AGTTCCTCCTGCTCCATCAAATG |
| tcdB | F01 | AGGTGCAGCAATCAAAGAGCTAA |
| | R02 | TGATGGTGCTGAAAAGAAGTGATC |
| cdtA | F739A | GGGAAGCACTATATTAAAGCAGAAGC |
| | F739B | GGGAAACATTATATTAAAGCAGAAGC |
| | R958 | CTGGGTTAGGATTATTTACTGGACCA |
| cdtB | F617 | TTGACCCAAAGTTGATGTCTGATTG |
| | R878 | CGGATCTCTTGCTTCAGTCTTTATAG |
| | F252; | CATGGTTCAAAATGAAAGACGAC |
| tcdC | R415 | GGTCATAAGTAATACCAGTATCATATC |
| | | CTTTC |

### Example 2

### Identification of C. difficile Ribotype 027 by Using a Pair of Specific Primers

After a comparison and analysis of the genome sequences of C. difficile CD196 (ribotype 027) (NCBI Reference Sequence: NC 013315.1) and C. difficile 630 (ribotype 012) (NCBI Reference Sequence: NC_{—}009089.1), the present inventors found that there was a gene region (target gene, SEQ ID NO:1) specific to ribotype 027 existed in the genome of C. difficile CD196. This gene region ranging from 3,490,544 to 3,493,524 mainly comprises CD196_{—}2946 gene and CD196 2947 gene which are respectively coding for hypothetical protein and CRISPR-associated helicase Cas3. Therefore, a pair of primers was specifically designed according to this target sequence for purpose of being a marker to detect ribotype 027 in clinical practice. Primers unique to ribotype 027 used for PCR were a forward #1 primer and a reverse #2 primer. The forward #1 primer and the reverse #2 primer can be a sequence of SEQ ID NO:2 (24 bp, Tm: 57.1° C.) and a sequence of SEQ ID NO:3 (19 bp, Tm: 51.1° C.), respectively, which can be used to amplify the length of 2,981 bp.

After using the primers for alignment with genomes of 17 sequenced strains of C. difficile, including CD169, BI-1, R20291, 2007855, CIP 107932, QCD-37x79, QCD-97b34, QCD-76w55, QCD-66c26, QCD-32q58, 630, M68, M120, QCD23m63, ATCC 43255, B1-9, and QCD-63q42, provided by the database of National Center for Biotechnology Information (NCBI), the present inventors found that this unique target gene was only existed in ribotype 027 strains.

DNA extraction of 17 strains were performed to conduct PCR by the forward #1 primer (20 pmol), the reverse #2 primer (20 pmol), and Phusion Flash PCR Master Mix (Thermo Scientific). The parameters for the amplification were listed in the followings, including: (1) denature template at 98° C. for 10 sec; (2) amplify template for 30 cycles of 1 sec at 98° C., 5 sec at 54° C., and 70 sec at 72° C.; and (3) extension (or elongation) at 72° C. for 1 min; the final PCR products were stored at 4° C. A 1% agarose gel was used for DNA electrophoresis (100 volts for 50 min), and then the gel was stained with ethidium bromid (EtBr) for analysis. Acquired products having the same sequence or the length (2,981 bp) of the sequence as SEQ ID NO:1 indicate that specimens comprises C. difficile ribotype 027.

Result

Result 1: The Pair of Primers (#1 and #2) is Specific to C. difficile Ribotype 027.

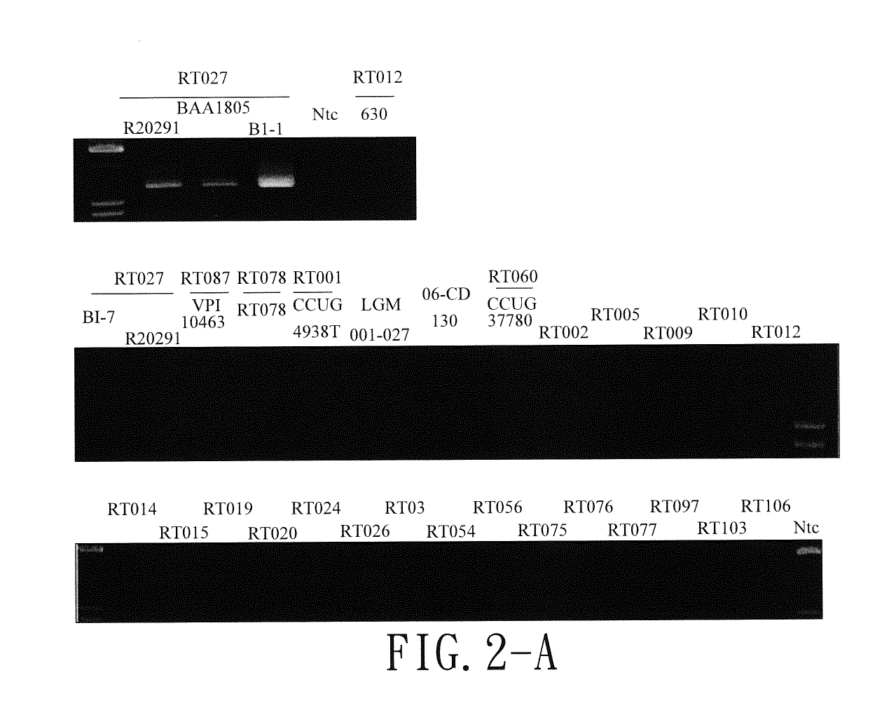

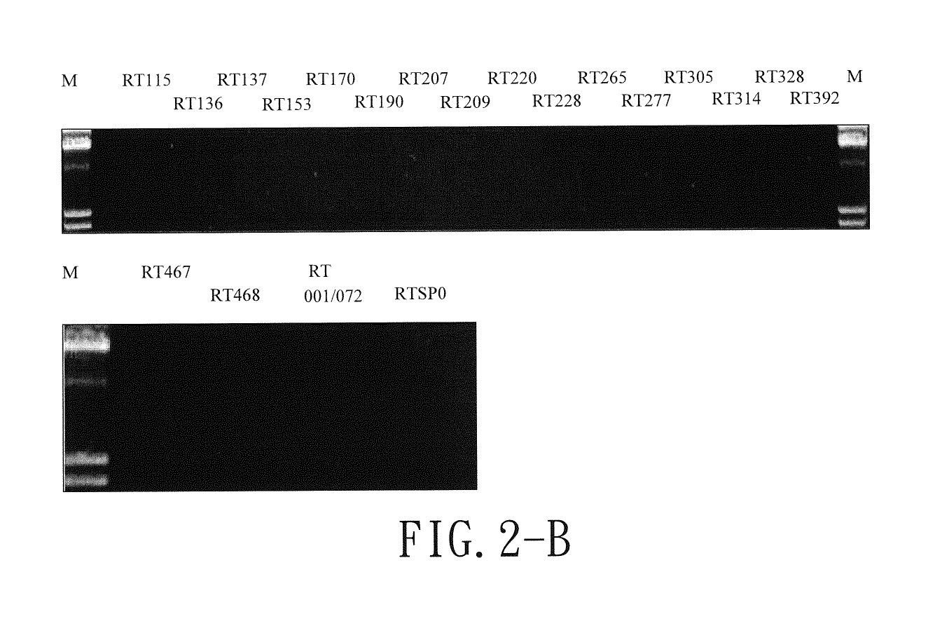

Referring to FIG. 2-A and FIG. 2-B, electropherograms respectively showing the ribotyping results of thirty-one standard strains and twenty standard strains are revealed. Among these strains with known ribotype, only four of ribotype 027 strains (R20291, BAA 1805, BI-1, and BI-7) have a DNA product consistent with the fragment size of the target sequence. It is worth mentioning that ribotype 027-related strains, i.e. RT019, RT075, and RT153, don't show any DNA product consistent with the fragment size of the target sequence.

In addition, toxin genotyping was conducted in the collected 414 clinical strains for a preliminary screening and a toxin typing classification. Clinical strains classified in different toxin groups, especially those strains with high virulence genotypes, were then selected to conduct multi locus sequence typing (MLST) for advanced sub-categories so as to acquire various groups having similar characteristics. It is worth mentioning that ribotypes of these clinical strains were all determined in advanced, including ribotype 027-related strains, ribotype 078 strains, ribotype 078-like strains and so on (data not shown). These 414 clinical strains were further used for detection by means of the present method and the technique platform. Two ribotype 027 strains, BAA1805 and R20291, were used as positive controls.

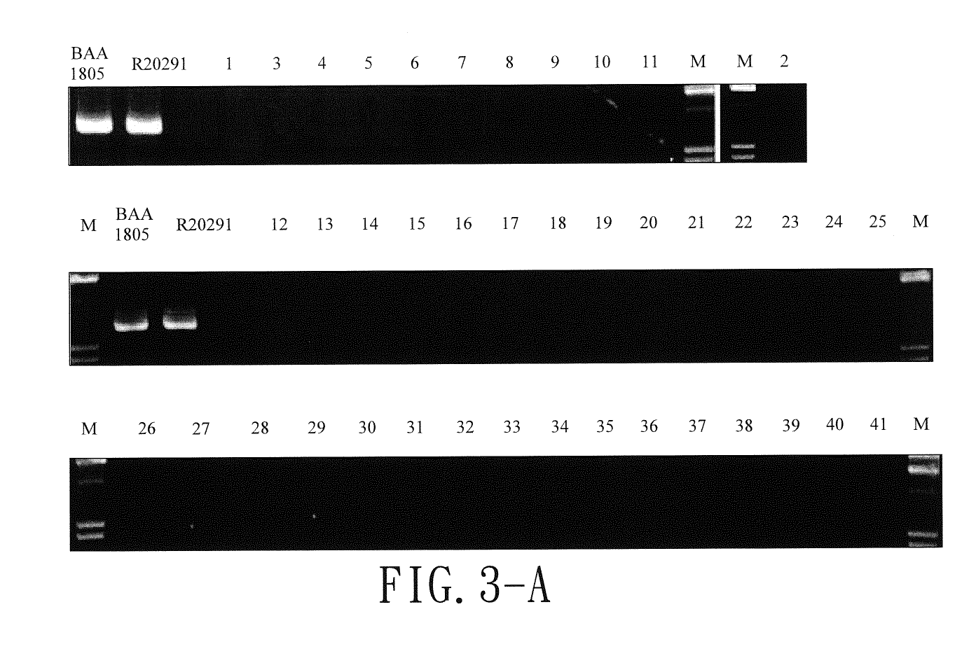

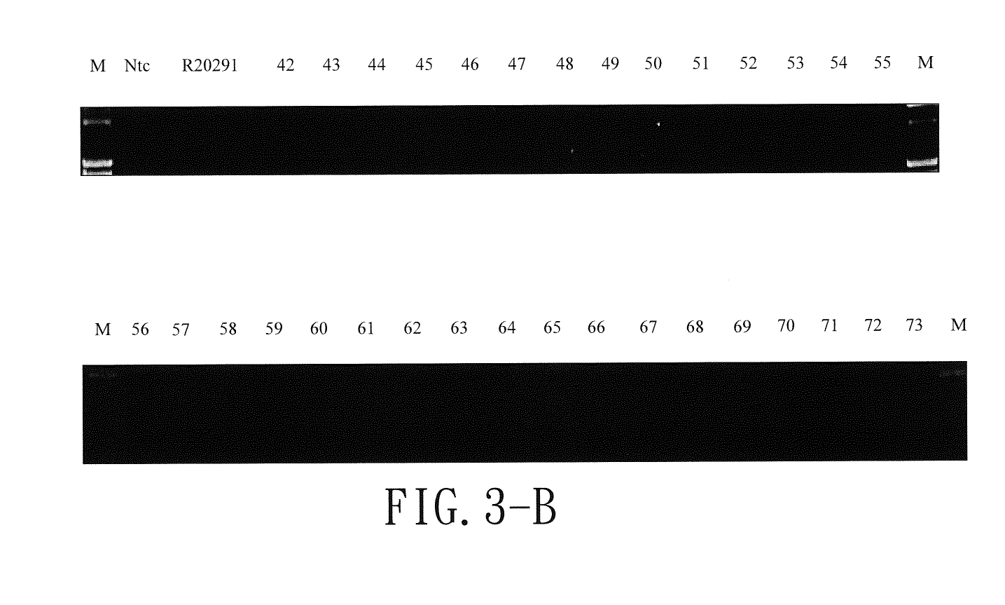

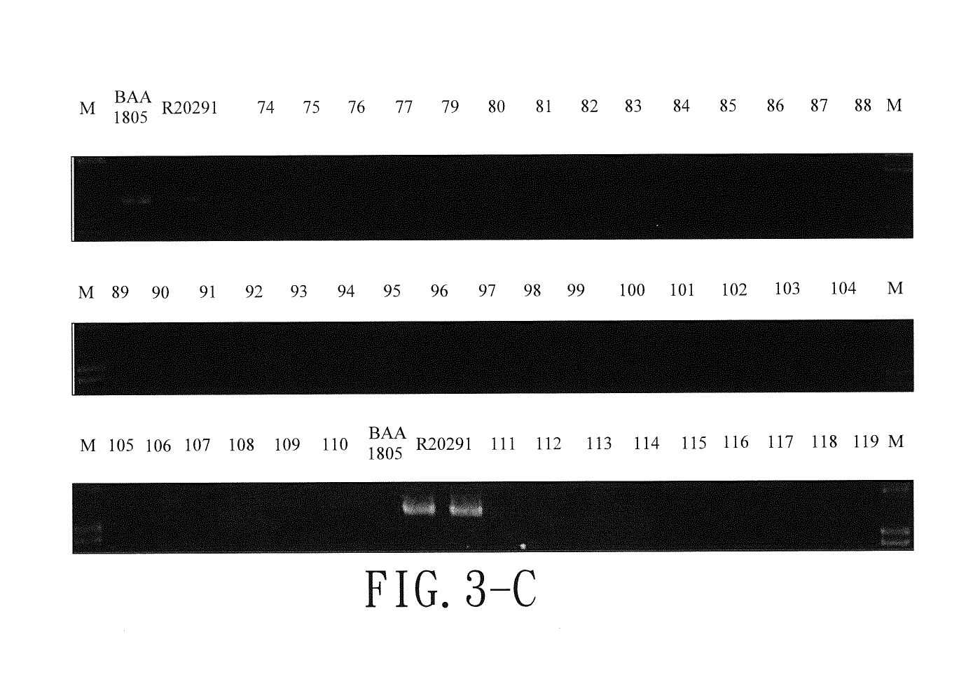

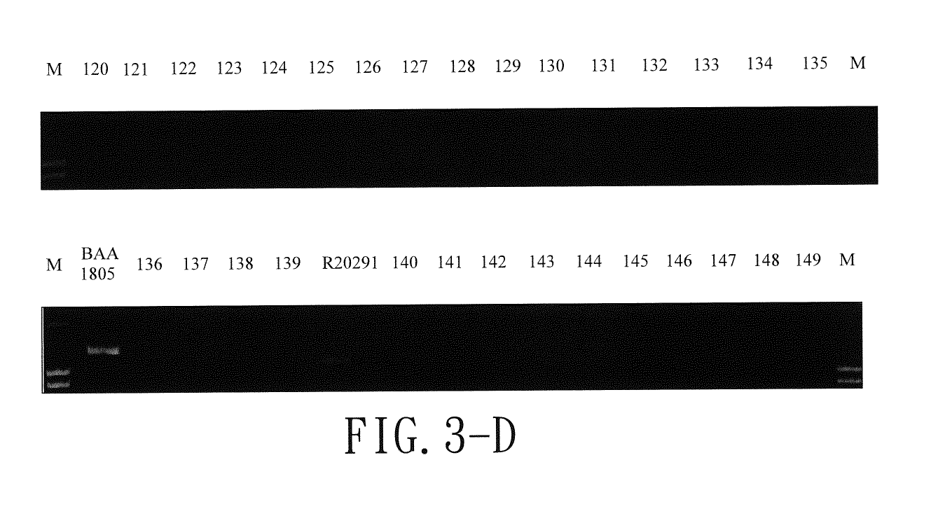

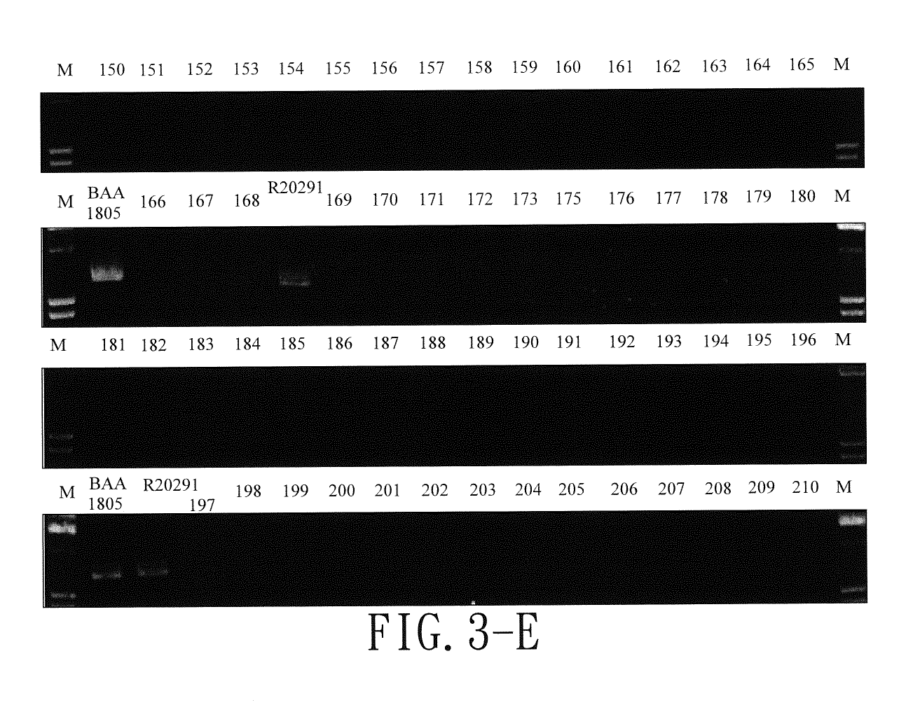

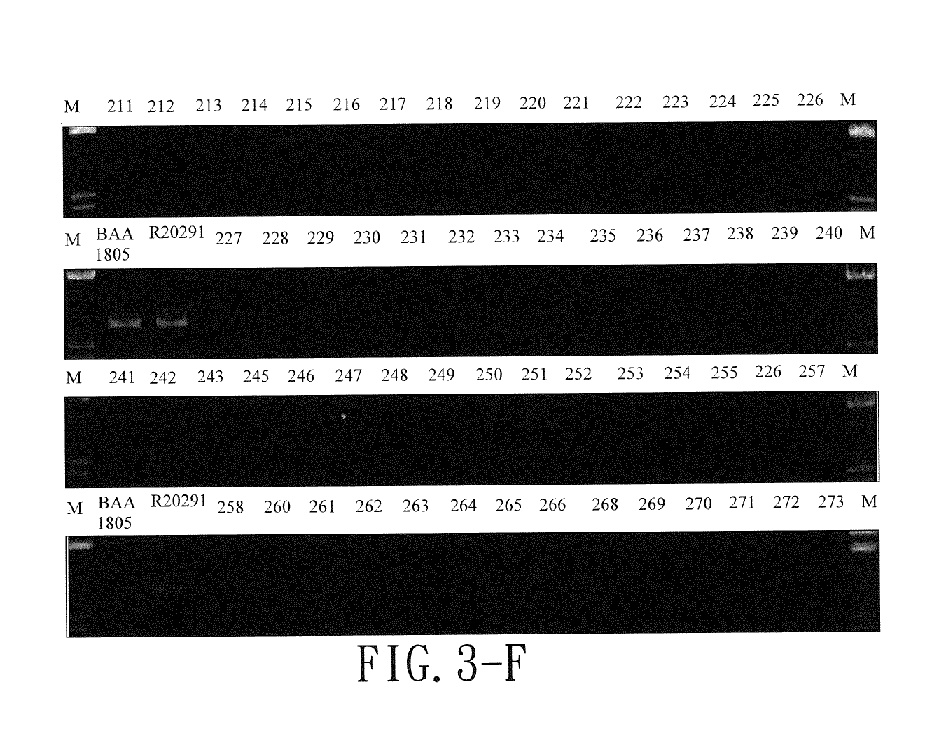

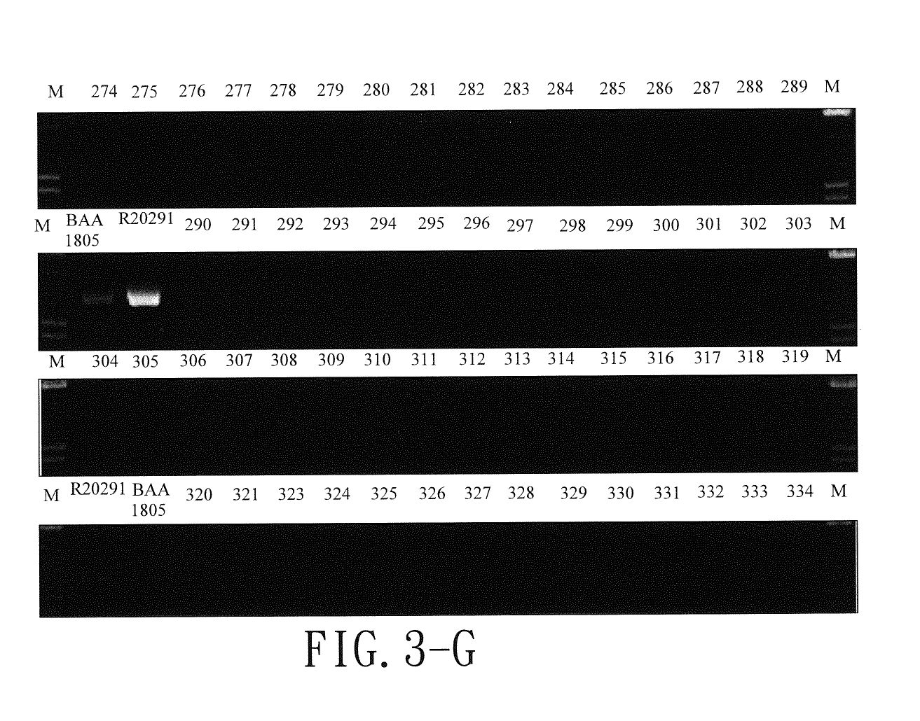

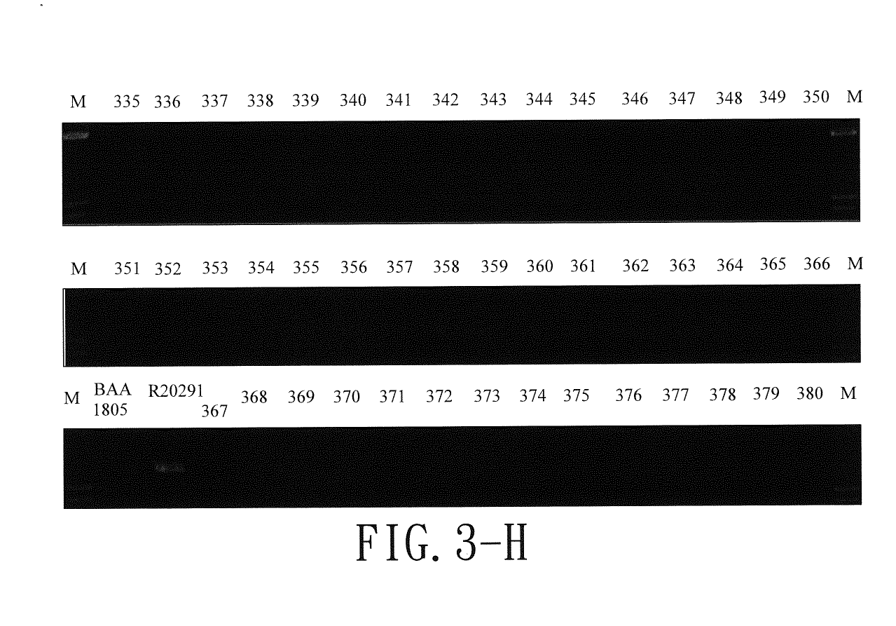

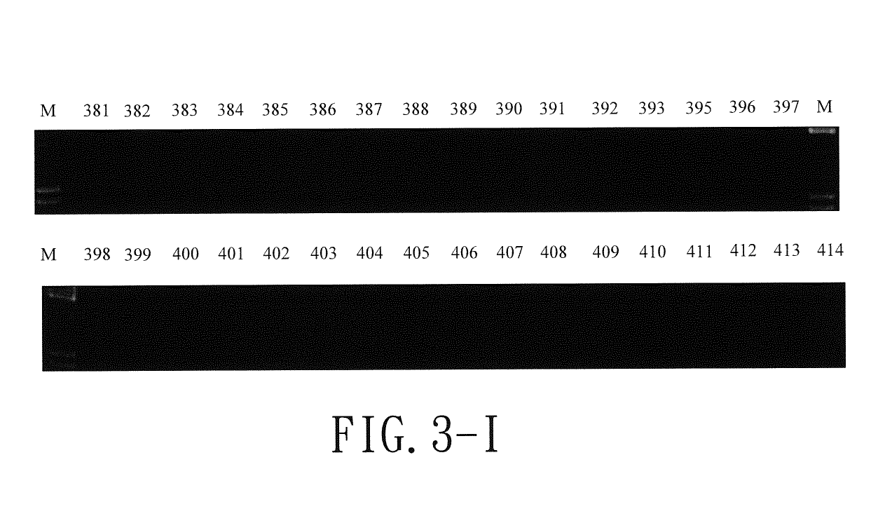

The results as shown in FIG. 3-A-FIG. 3-I, only ribotype 027 strains (BAA 1805 and R20291) have a DNA product consistent with the fragment size of the target sequence. This result was consistent with the previously known results.

Result 2: The Pair of Primers (#1 and #2) can be Used to Distinguish Ribotype 027 Strains from Ribotype 027-Related Strains.

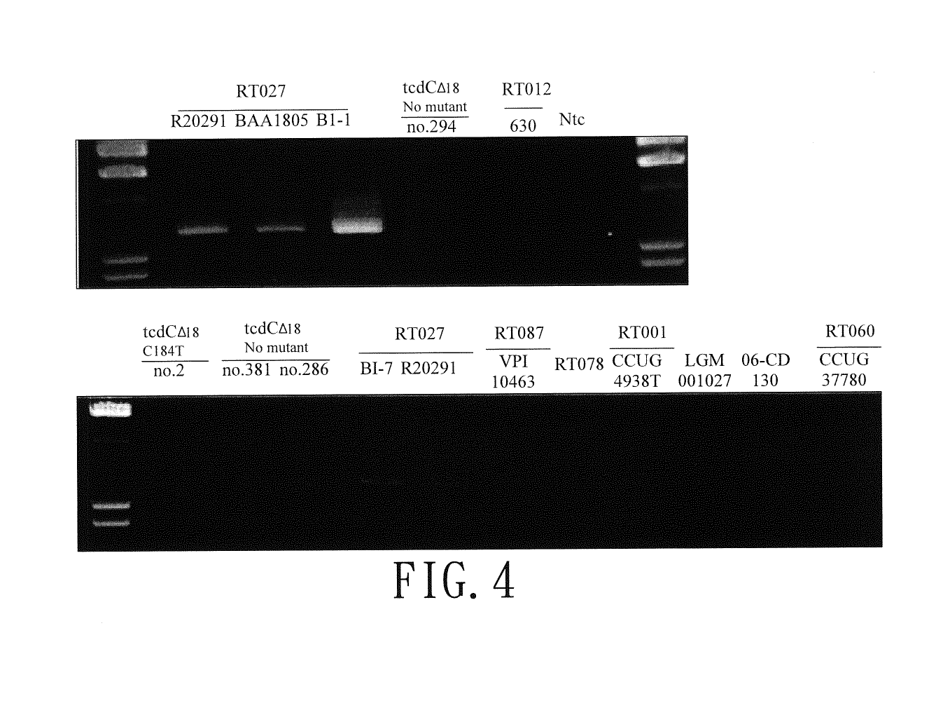

Although ribotype 027 strains are characterized by having an 18 bases deletion in negative regulator tcdC gene, strains with a deletion in tcdC gene do not all belong to ribotype 027. Therefore, in order to avoid false positive results, the inventors found that there were four strains with 18 bases deletion in negative regulator tcdC gene (as no. 294, 2, 381 and 286 of FIG. 4). After further MLST and ribotyping analysis, it was proved that these four strains were not ribotype 027 strains. Accordingly, using primers specific to ribotype 027 according to the present invention can accurately distinguish the ribotyping 027 strains (only ribotype 027 strains including R20291, BAA 1805, BI-1, and BI-7 have DNA products consistent with the fragment size of the target sequence) without false positive results of detection. Therefore, the primer pair (#1 and #2 primers) and the target sequence are indeed with specificity for the detection of ribotype 027, which can provide a rapid and simple diagnostic method for clinical practice.

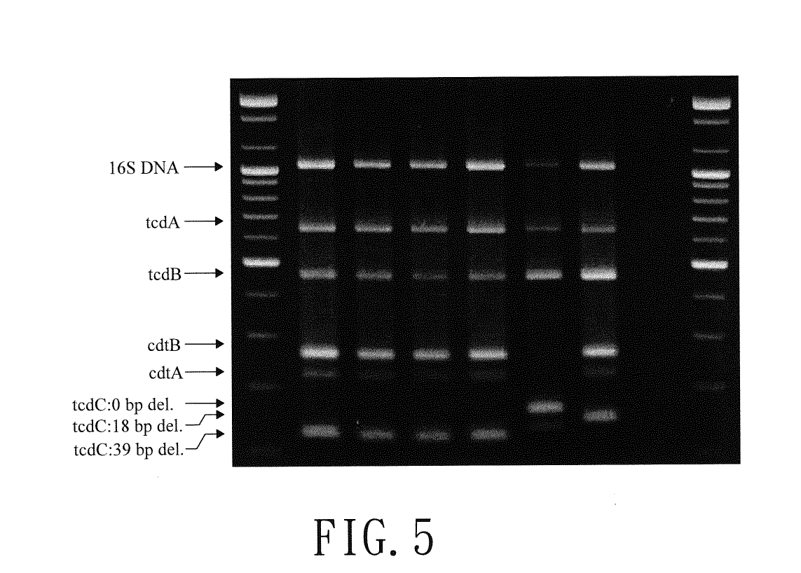

Referring to FIG. 5, an embodiment of a toxin genotyping result of C. difficile is revealed. The present inventors have also set up a multiplex polymerase chain reaction (multiplex-PCR) for fast screening virulence genotypes of clinical strains. It mainly comprises six genes, i.e. 16s rDNA, tcdA, tcdB, cdtA, cdtB, and tcdC, as a basis for toxin genotyping of C. difficile. Therefore, the result can be used to rapidly detect toxigenic C. difficile.

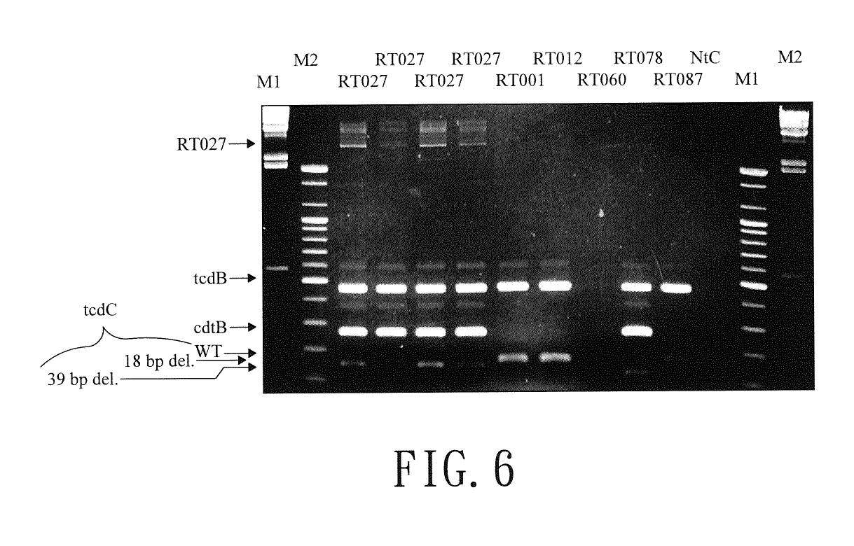

Referring to FIG. 6, an electropherogram showing the results of toxin genotyping and ribotyping of different C. difficile strains is disclosed. In summary, a technique platform for conducting multiplex-PCR has been established for quick screening virulence genotypes of clinical strains, which was mainly based on three genes including tcdB, cdtB, and tcdC, plus a gene unique to ribotype 027. Accordingly, the technique platform can be developed into a single step and a sensitive technology platform to be a basis for toxin genotyping and ribotype 027. The #1 and #2 primers of the present invention can be used to specifically detect whether the specimen is a C. difficile ribotype 027 strain. Therefore, the combination of virulence genotyping (for rapid screening toxic C. difficile) and the pair of primers (specific to the target sequence) will significantly improve the efficiency of detecting C. difficile ribotype 027.

According to the above description, in comparison with the traditional technique, a sequence, a technique platform, and a method for in vitro detecting C. difficile ribotype 027 according to the present invention has the advantages as following:
1. With primers designed for a target sequence unique to ribotype 027, the present invention only requires one positive strain as a reference and a gene amplification process, so it solves the disadvantages of the traditional methods, e.g. necessity for standard strain in advance and requirement of a powerful software and computer for extraordinary analysis. Moreover, the results can be directly acquired from the images of electrophoresis gels, so the present invention also has advantages of high specificity and simple process for detecting C. difficile ribotype 027.
2. By obtaining a specimen DNA for in vitro detecting whether the specimen is ribotype 027 or not, the present invention not only can develop into a commercial kit for rapid screening but also exhibit the potential for being a one-step, rapid and sensitive diagnostic alternative for the detection of C. difficile ribotype 027.
3. Currently there are no diagnostic techniques for directly detecting ribotype 027 strain on the market. The technique platform and the method thereof that can directly and quickly detect ribotype 027 will significantly save patient's time for waiting the test results so that patients can get the proper treatment instantly.

What is claimed is:

1. An purified and isolated target sequence for in vitro detecting C. difficile ribotype 027 comprises at least 85% sequence homology with SEQ ID NO: 1.

2. The purified and isolated target sequence for in vitro detecting C. difficile ribotype 027 as claimed in claim 1, further has a DNA sequence comprising at least 90% sequence homology with SEQ ID NO:1.

3. The purified and isolated target sequence for in vitro detecting C. difficile ribotype 027 as claimed in claim 2, further has a DNA sequence comprising at least 95% sequence homology with SEQ ID NO:1.

4. A technique platform for in vitro detecting C. difficile ribotype 027 comprises:
a pair of primers including a #1 primer and a #2 primer specific to C. difficile ribotype 027 for conducting polymerase chain reaction (PCR); and
primer sets and materials demanded for multiplex-PCR.

5. The technique platform for in vitro detecting C. difficile ribotype 027 as claimed in claim 4, wherein the #1 primer includes a sequence of SEQ ID NO:2 and the #2 primer includes a sequence of SEQ ID NO:3.

6. The technique platform for in vitro detecting C. difficile ribotype 027 as claimed in claim 4, wherein the #1 primer and the #2 primer are designed for the sequence of SEQ ID NO:1 of C. difficile ribotype 027.

7. A method for in vitro detecting C. difficile ribotype 027 comprises the steps of:
(A) obtaining a specimen DNA; and
(B) in vitro amplifying the specimen DNA by primer sets to detect whether the specimen is matched to characteristics of a target sequence of SEQ ID NO: 1, where the specimen is determined to comprise C. difficile ribotype 027 on a condition of its sequence matching to characteristics of the target sequence.

8. The method for in vitro detecting C. difficile ribotype 027 as claimed in claim 7, the specimen in step (A) is identified as C. difficile, with C. difficile toxins or a combination thereof.

9. The method for in vitro detecting C. difficile ribotype 027 as claimed in claim 7, wherein the specimen is selected from infected stools or C. difficile isolates.

10. The method for in vitro detecting C. difficile ribotype 027 as claimed in claim 7, the characteristic of the target sequence of SEQ ID NO: 1 comprises a sequence or a length of the sequence.


SEQUENCE LISTING (3 sequences)

SEQ ID NO: 1 - DNA, 2981 bp, Clostridium difficile
caataaatattgtgtcttttgaaatttcattattatctccaagaaagtttgtaaactgtgcaatcaagcttttgcctattagttaaaaatgtatactgcgaaattttgcaattgttgaatggtctggagtggaggtttcgcctagaaggtacataaaattaatatctcttttgcatgttctttctatttctctcgaagaataaatacagttcatatctgtatatattataactttcaaaaattgtctatggattactctatttcatctaattcgagaataagtcttatataatttttctaaattcatctcctcaataatttaacttaataaccttacagaatcatcttttggaatcatgtaatcaatatttagtggtagcttaaattgataattatcaccataagtagtataatcaatttgtgtagtattttgttttagcatacagttattatacaagaatcccaggttcttttgaacttgggatttatttttttatacaaaaaagagcgtagcactattttctagtgcgacagcctttttatttatataaccttctagttgtgtgctaattgtgtgcttttaagcaaaataaaaggaattacttagtccaaacataagtaattccttttattctattattttcattttcaaaattggtggagacgaggggagtctacaagtggttatttgattgttttgttggttttcgtaagtgtaggtatatagctatttagttatgatgttatactaatttttttggatattttttattatttatttttgtggttggggtattattggggtataattttctactttctaaaactcttgctaattcaataatataaatcaaactctcagttaagtttcttaatgaataagtaaatttacaactaatttagtgtaatctaaacttgtagtgcctagtcacaactctcaccccttcaataatttataactaacttagtgtaatttaaacctctctactaaaattacttccacatatagctacagaatttataactaacttagtgtaatttaaactgtacctgggtcttttatcttacttgtaagtgtacttctatttataactaacttagtgtaatttaaacattgcagataggtctgtgccatcattcgtgctgttacatttataactaacttagtgtaatttaaactgggatggattttgaagcttctatgtcaaatgtacaatttataactaacttagtgtaatttaaacgaaacctctctttaaaattattcatagaatttataaaatttataactaatttagtgtaatttaaacattgttgctagaaaacattaaaatacattacacataatttataactaacttagtgtaatttaaacggctggacttgtaatagttgtaactaaaattagtgaaatttataactaacttagtgtaatttaaacgggtagaatgaatcaaatagcaaaagtactttccggaatatttataactaacttagtgtaatttaaaccccaaataaaactatagtaattacaacacttgtagggttattaaaatagttaatttgcagtgaggcaccagtagtgtttttagctaaaaacaaaaattcagttataaaccttgccatcactaactcacagccatttacacatatttttcgctcactgcaaaattattctattgtaacatataaaatttgtagtcgatataaattataacaaattctcaataaaatattttaattagttatactataattctcttaacaatcaaatattagacctagtttatcattataactataacttgtaacaactatatctccacattcataaaacatctttttattataattgtatctttttacagatacatttacaaataatgaactcgcttctaaatacatttttttatcaatatacaacttgtactcttctaaaagacattcaggcacaacactaatagtatcattatttaacatatcattattagcagatttattccatacccctaacctcatatttttcattataatctctaaagcaactttatgtttattatatttatccatatctatatctaaatagacattatctaaataagatggaactttactctcatcaactatagaattatcattatgtattatatctgatattactatatcagtcctatttattatatcttcatcatatatattaagctttctatcataatcatatatataaatattgctcaactcagcattatctccccttctatttactctaccaaacctttgtaacaatgaatctaaaggagctaactctgtataaagaacatcataacttatatctaaacttacctctatactttgagtacctatcaataagaagctttcttctatccctttttctgcctgctctctatctctagtattaaatcgaccatgtattaatttaggattatatttttgaagccttccatataactcttgagataaatctactgaattaacacatattaatactctttttccttgcattaaatcattttctattttatgtatgctatcaagtattccttcacttaatctattcaatctatgtctaaatattaaatctttactatctggttcaataattgtacttatttgcaatctttcctgtattaacttttttaagcaagttggtatacttgcactcattatacatatttttacatttaagttttcttttaaatattttaaaaattccaatgtataacacaatactcttgtatcaaaacaatgtatttcatcaactataaatatactatcttttaattgtgatagtagcatttcaaatctcttacaagaaaatactgactttactaattgaaatatagtacatacattaatttgttttacactctttttgtatatctggtaaagttgttttatatcttcttcatcactaattttattcaaaaaatatggagctttactgtgtaacatacttacgc

SEQ ID NO: 2 - DNA, 24 bp, Artificial sequence
  Synthetic
caataaatattgtgtcttttgaaa

SEQ ID NO: 3 - DNA, 19 bp, Artificial sequence
  Synthetic
gcgtaagtatgttacacag